(19) United States
(12) Patent Application Publication
(10) Pub. No.: US 2016/0095799 A1
(43) Pub. Date: Apr. 7, 2016

(54) METHOD FOR MAKING A MACHINABLE BLOCK OF POLYURETHANE ELASTOMER FOR USE IN FABRICATING DENTAL APPLIANCES

(71) Applicants: Li Luo Skelton, San Antonio, TX (US); Danielle Ruggiero, Canton, GA (US)

(72) Inventors: Li Luo Skelton, San Antonio, TX (US); Danielle Ruggiero, Canton, GA (US)

(21) Appl. No.: 14/723,497

(22) Filed: May 28, 2015

Related U.S. Application Data
(60) Provisional application No. 62/059,281, filed on Oct. 3, 2014.

(51) Int. Cl. A61K 6/09; A61C 5/10; A61C 13/08; A61C 8/00; A61C 1/08; A61C 13/00; A61C 13/01

(52) U.S. Cl. CPC A61K 6/09; A61C 13/0022; A61C 5/10; A61C 13/01; A61C 8/0012; A61C 1/082; A61C 13/08

(57) ABSTRACT

A method of making a machinable puck of a hard, solvent free, substantially non-hydrophilic polyurethane elastomer for use in fabricating dental appliances. The elastomer has a hardness of not less than about Shore D65. A first polyester or polyether polyol with a functionality greater than two and a molecular weight greater than one hundred is reacted with an isocyanate prepolymer prepared from one or more branched polyester or polyether polyols with a functionality greater than two; and then blending prepolymer and first polyester or polyether polyol with nanoparticles and colorants as needed. In one embodiment, the polypropylene glycol triether and the isocyanate prepolymer comprises dicyclohexylmethane 4,4′-diisocyanate. The machined, elastomer appliance is able to be heated in hot water to make it flexible and formable for individual, customed-fitting for the patient.

### PRIORITY

This application claims priority to copending U.S. Provisional Patent Application Ser. No. 62059281, filed Oct. 3, 2014 and copending U.S. Utility patent application Ser. No. 14/602,343, filed Jan. 22, 2015, both of which are incorporated herein for all purposes.

### BACKGROUND OF THE INVENTION

This invention relates to a method of making a machinable block, puck, or disc of a hard, shock-absorbing polyurethane elastomer for use in fabricating dental appliances. More particularly, a machinable dental block of the elastomer has a hardness of not less than about Shore D65 and is made by reacting a first polyester or polyether polyol with an isocyanate prepolymer and blending them with nanoparticles and colorants (as needed); allowing the block to cure; and then milling or machining the block via a programmable milling machine to form the ultimate jacket, appliance, prosthesis, or restoration.

Dental appliances, as that term is used herein includes dental jackets (customized or pre-made) full or partial dentures, full or partial implant dentures, fixed or removable implant bridges, implant crowns, Maryland bridges, surgical guides, milling blocks, and individual false teeth that may be fixed or removable replacements for teeth. Tooth replacement becomes necessary when there is single or multiple tooth loss, full arch and/or full mouth tooth loss or roots have been irreparably damaged, and the tooth has been lost or must be removed. These losses or damage may be due to many causes, including but not limited to, genetic factors; economic difficulties; poor education; neglected and poor diet and hygiene; systemic related tooth loss (such as autoimmune disease); aging and excess wear due to stress; advanced periodontal disease and caries; and failing crowns, bridges, or other prostheses.

Dentists have long known that a missing permanent tooth should always be replaced or else the teeth on either side of the space gradually tilt toward the gap, and the teeth in the opposite jaw begin to move toward the space. Dentists have also known that missing teeth may result in not only physical damage, but also mental and psychological damage.

Generally speaking, the adverse effects of tooth and teeth loss may be summarized as including bone loss; loss of chewing power and chewing function; loss of facial esthetic; loss of the stability of dentition for single or multiple teeth loss; loss of occlusion stability thereby resulting in TMJ issues; loss of phonics resulting in speech problems; loss of self-confidence; poor digestion and nutrition; and instability of the entire spine and pelvis complex due to missing teeth causing occlusal problems.

There are several standard forms of tooth replacement in modern dentistry. Dentures are false removable teeth that are relatively quick and easy to fabricate. They may be partial, where only one or a few teeth are replaced; or they may be complete, where all the teeth are missing in the jaw and are replaced.

A partial denture holds false teeth on a plastic or metal framework. The framework is removable, and is designed to fit around the adjacent teeth. It may cover part of the gum tissue or roof of the mouth. There may also be hooks and rests to help hold or support the framework. Sometimes these hooks or rests are visible when smiling. Dentures are normally removed for nightly cleaning and to allow the gum tissue to breathe.

Another form of tooth replacement is the bridge. A bridge uses natural teeth adjacent to an empty space to support a false tooth/teeth there between. The natural teeth usually need to be shaved or contoured so that a crown (cap) may be placed over the top of the natural teeth. These are called abutment teeth. The crowns on each tooth support the false tooth/teeth there between, known as the pontic tooth/teeth.

Dental implants may also replace natural teeth. Implants are usually composed of two major components: the implant which acts as an anchor in the jaw; and the crown which is the tooth part visible in the mouth. Implants are excellent options for tooth replacements as they do not affect adjacent teeth and yet look, feel, and function as natural teeth do.

An overdenture may be used when the natural root of a tooth is preserved to prevent or delay bone loss that occurs when the entire tooth is removed. The dental professional bonds metal attachements to the root of the tooth and then snaps an overdenture into them. The remaining roots actually provide increased sensation to the wearer of the overdenture and the dentures feel more similar to natural teeth.

Some patients do not like the feeling of a denture on the roof of the mouth or palate. These patients experience a gagging sensation and find swallowing (and even speaking) difficult with a full palate denture. A palate-less, snap on denture is one solution to this situation. In order for a palate-less snap on denture to function properly, it must be snapped into 4-5 implants depending on the size of the patient's arch. Plate-less snap on dentures are a favorite among patients because of the higher comfort level associated with very secure and stable, teeth

The All-on-4® treatment concept provides edentulous and soon-to-be edentulous patients with a fixed full-arch prosthesis on four implants on the day of the surgery.

A new full mouth implant treatment has been developed by one of the inventors herein which provides and instant smile and function make-over. This unique treatment features utilization of a unique and inventive polyurethane elastomer and incorporates a holistic and lifelong oriented support program. The full mouth implant uses six implants to support a full arch, detachable, fixed nano-polymer bridge.

Historically, a variety of materials have been used to replace lost teeth. Animal teeth and pieces of bone were among the earliest of these primitive replacement materials. In the last few hundred years, artificial teeth have been fashioned from natural substances such as ivory, porcelain, and even platinum. These comparatively crude prototypes of earlier times were carved or forged by hand in an attempt to mimic the appearance and function of natural teeth.

Modern technology has offered considerable advances in the materials used to make artificial teeth and improved techniques for affixing them in the mouth. Synthetic plastic resins and lightweight metal alloys have made teeth more durable and natural looking. Better design has resulted in dentures that provide more comfortable and efficient chewing.

Most artificial teeth are made from high quality acrylic resins bonded to an acrylic base, which make them stronger and more attractive than was once possible. The acrylic resins are relatively wear-resistant, and teeth made from these materials are expected to last between five and eight years. The main problems with such artificial teeth occur with the acrylic base dental appliance, especially the implant support prosthesis. The tooth/teeth/pop out from the base and there is cracking and chipping of the teeth. Further the appliance may easily break causing considerable injury to the wearer.

A flexible-base nylon dental appliance provides a strong and light, metal free partial denture, but its major disadvantage is its inability to be relined or added onto. Such flexible-base nylon are difficult to manufacture and require the use of special thermal injection machinery.

Porcelain is also used as a tooth material because it looks more like natural tooth enamel. Porcelain is used particularly for upper front teeth, which are the most visible. However, the pressure of biting and chewing with porcelain teeth can wear away and damage natural teeth. Further, porcelain provides no shock absorption function. This places more bite forces on other components of the dental appliance, especially underneath supported bone. Even with high quality acrylics and porcelains used today, cracking and chipping of the teeth continues to be a significant problem. With dental appliances made of these current compositions, the tremendous forces created by the wearer during mastication may be transferred through the appliance to the muscles and bone. The appliance may not crack, chip, or break before causing considerable injury to the wearer. There continues to be a need for a dental material which is hard and strong but with flexibility to absorb and yield without cracking and breaking.

Every individual's mouth is different, and each dental appliance must be custom designed to individually fit for functionality and still be cosmetically effective. The latest methodology used in denture design, known as dentogenics, has developed standards for designing teeth to fit specific smile lines, mouth shapes, and personalities. These standards are based on such factors as mouth size and shape, skull size, age, sex, skin color, and hair color. For example, through proper appliance design, patients can be given a younger smile by simply making teeth longer than they normally would be at that patient's age. This rejuvenation effect is possible because a person's teeth wear down over time; slightly increasing the length of the front teeth can create a more youthful appearance.

The unique combination of hardness, flexural strength, and thermal elastomer properties of the present inventive dental block composition results in a hard, shock-absorbing polyurethane elastomer which solves the problems associated with present materials and appliances. The present composition allows for an ideal, moldable, premade dental jacket which maintains the principle of dentogenics, but also provides a clinic with a simple procedure for rapid production, allowing for one-visit treatment. These developments have been made possible by the improvements to the material used to make the dental appliances.

Computer aided manufacturing (CAD/CAM) technology has made significant strides in the field of dentistry. With the CAD/CAM process it is possible to scan the denture base morphology and tooth positions recorded with this technique and import those data into a virtual tooth arrangement program where teeth can be articulated and then export the data to a milling device for fabrication of the complete denture.

Because the denture base is fabricated by machining, polymerization shrinkage of the resin is eliminated, and the fit of the denture base is superior to that of conventionally fabricated denture bases. The polyurethane elastomer disclosed herein has been found to be a superior composition for machining the denture from blocks, discs, or pucks of the elastomer.

### SUMMARY OF THE INVENTION

The present invention is a method of making machinable dental blocks, discs, and pucks of a hard, solvent free, substantially non-hydrophilic polyurethane elastomer having a hardness of not less than about Shore D65 by reacting a first polyester polyol with a functionality greater than two and a molecular weight greater than one hundred with an isocyanate prepolymer (either aliphatic or aromatic) prepared from one or more branched polyester or polyether polyols with a functionality greater than two; blending said prepolymer and first polyester or polyether polyol with nanoparticles and appropriate colorants as needed. Using the elastomer pucks as a base for machining various dental appliances, including making customized or premade dental jackets, full and partial dentures, full and partial implant dentures, fixed and removable implant bridges, implant crowns, Maryland bridges, surgical guides, milling blocks, and replacement teeth.

### BRIEF DESCRIPTION OF THE DRAWINGS

There are no drawings in this disclosure.

### DETAILED DESCRIPTION OF THE PREFERRED EMBODIMENT

The following is a brief discussion of manufacturing processes for making a machinable dental block, puck, or disc for making a dental appliance. The blocks may be used for making customized or premade dental jackets, full and partial dentures, full and partial implant dentures, fixed and removable implant bridges, implant crowns, Maryland bridges, surgical guides, milling blocks, and replacement teeth.

Data is programmed into a CAD/CAM machine which controls a milling machine which will create the actual dental appliance from the machinable dental block. The process of machining dental appliances using CAD/CAM technology is well known and is not the unique aspect of the present invention.

Because of the number of processing steps there may be a slight discrepancy in the fit. Usually just a minor grinding and smoothing of surfaces is all that is necessary to make the machined denture fit correctly.

Further, with the polyurethane elastomer disclosed herein, minor adjustments may be made by placing the machined denture or jacket in hot water (approximate range 150°-210° F., preferably about 180° F.) to make the denture or jacket more flexible and formable.

Further yet, it has been found with the use of the polyurethane elastomer disclosed herein a plurality of standard sized dentures may be made available to fit a variety of most common patient mouth sizes.

An embodiment of the method of manufacturing prosthetic teeth has been developed using a two component, solvent free, nanoresin composite polyurethane system. This two component polyurethane system consists of an isocyanate terminated prepolymer and a polyol blend. The polyurethane system incorporates a polyol, isocyanate, and nanoparticles and colorants as needed.

The nanoparticles used may be of various sizes, makeup, and density. The nanoparticles may be, but not limited to, barium glass, strontium glass, and aluminum oxide. The varying particle size of the nanos may be between 0.5 to 15 micrometers.

The composition of the polyol blend consists of a polyester with functionality greater than two and, a molecular weight greater than one hundred. The polyol blend that reacts with an isocyanate prepolymer prepared from one or more branched polyester or polyether polyols. The hydroxyl weight of the polyols may range from 56 to 400, preferably 200 to 600. The polyether polyols may consist of polyfunctional alcohols such as glycerine, trimethylolpropane, pentaerythritol or polytetramethylene glycol. They may be polyether diols or triols with either ethylene oxide or propylene oxide end caps. The polyester polyols may be, but not limited to, dibasic acid reacted with a diol such as ethylene glycol, 1,2-propylene glycol, or diethylene glycol.

The polyol blend may be also composed of additives such as surfactants, moisture scavengers, antifoam additives, pigments, and stabilizers. Pigments or pigment dispersions of multiple colors may be used. They may be composed of, but not limited to, titanium dioxide, calcium carbonate, iron oxide, and the like dispersed in the polyether polyol.

The isocyanate may be either aliphatic or aromatic. Aromatic polyisocyanates may be, but not limited to, 2,4-tolune diisocyanate and 4,4′-diisocyanate. The aliphatic polyisocyanates may be, but not limited to, a hexamethylene diisocyanate, dicyclohexylmethane, 4,4′-diisocyanate, or isophorone diisocyanate. The isocyanate terminated prepolymer may be formed by reacting polyols with isocyanate with an excess of isocyanate. Functionality of the prepolymer itself should be greater than two.

This final composite structure is a reaction of the prepolymer with a polyol mixture in a volume or weight ration of the prepolymer and polyol blend of 1:1; and, therefore, is a polyurethane system providing a Shore D hardness of greater than 65.

Thus, the present inventive polyurethane dental material may be molded into a machinable puck from which a dental appliance may be machined.

In the above discussion of one embodiment of the manufacturing process, the step of filling the machinable dental mold with the elastomer composition is an important step. The present inventive method may utilize an injection gun which allows the technician to have side-by-side cartridges containing the two components of the elastomer in liquid form.

One cartridge contains a composition of a polyether polyol or polyester polyol with a functionality greater than two and which has a molecular weight greater than one hundred. One such composition is glycerol polypropylene glycol triether. The other cartridge contains a composition of an isocyanate prepolymer prepared from one or more branched polyester or polyether polyols with a functionality greater than two. The isocyanate may be either aliphatic or aromatic. One such composition is dicyclohexylmethane 4,4′-diisocyanate.

Application of a single action plunger will mix the components in a single stream in the proper proportions for use in filling the mold. Alternatively, each component may be initially in a “dry” form and converted to a liquid form as is known in the art. Each component may be mixed separately and then blended into one uniform liquid mixture for filling the mold. Colorants may be further blended into the composition to provide shading of the elastomer as needed

As previously stated, the composition of the dental appliance uses a two-component, solvent free, nanoresin composite polyurethane system. The polyurethane system incorporates a polyol isocyanate, and nanoparticles. The nanoparticles used may be of various sizes, makeup, and density.

The composition may be injected into an elongated, cylindrical mold, allowed to cure, removed from the mold, cut into appropriate sized disc or pucks, secured in a programmable milling machine, and then be milled via the programmable milling machine to form the ultimate dental appliance, prosthesis, or restoration. This embodiment relates to the CAD/CAM process for machining a final dental appliance.

It is to be understood that the invention is not to be limited to the exact details of operation or structure described as obvious modifications and equivalents will be apparent to one skilled in the art. The dental appliances herein can also be prepared employing any other known and conventional techniques known in the art.

We claim:

1. A method of making a machinable puck of a hard, solvent free, substantially non-hydrophilic polyurethane, elastomer having a hardness of not less than about Shore D65 for use in fabricating a dental appliance comprising:
reacting a first polyester or polyether polyol with a functionality greater than two and a molecular weight greater than one hundred with an isocyanate prepolymer prepared from one or more branched polyester or polyether polyols with a functionality greater than two;
blending said prepolymer and first polyester or polyether polyol with nanoparticles;
curing said elastomer in a mold;
removing said elastomer from said mold;
sizing said elastomer to a puck, block, or disc;
placing said puck into a programmable milling machine containing denture base morphology data; and
machining said puck to form said dental appliance.

2. The method of claim 1 wherein said isocyanate prepolymer is aliphatic.

3. The method of claim 1 wherein said isocyanate prepolymer is aromatic.

4. The method of claim 1 wherein said first polyester or polyether polyol comprises glycerol polypropylene glycol triether and said isocyanate prepolymer comprises dicyclohexylmethane 4,4′-diisocyanate.

5. A dental appliance of claim 1 wherein said appliance is selected from the group consisting of a customized or premade dental jacket, a full or partial denture, a full or partial implant denture, a fixed or removable implant bridge, an implant crown, a Maryland bridge, a surgical guide, a milling block, and a replacement tooth.

6. The dental appliance of claim 2 wherein said appliance is selected from the group consisting of a customized or premade dental jacket, a full or partial denture, a full or partial implant denture, a fixed or removable implant bridge, an implant crown, a Maryland bridge, a surgical guide, a milling block, and a replacement tooth.

7. The dental appliance of claim 3 wherein said appliance is selected from the group consisting of a customized or premade dental jacket, a full or partial denture, a full or partial implant denture, a fixed or removable implant bridge, an implant crown, a Maryland bridge, a surgical guide, a milling block, and a replacement tooth.

8. The dental appliance of claim 4 wherein said appliance is selected from the group consisting of a customized or premade dental jacket, a full or partial denture, a full or partial implant denture, a fixed or removable implant bridge, an implant crown, a Maryland bridge, a surgical guide, a milling block, and a replacement tooth.

9. The dental appliance of claim 1 wherein said appliance is a dental jacket.

10. The dental appliance of claim 4 wherein said appliance is a dental jacket.
